(19) United States
(12) Patent Application Publication
(10) Pub. No.: US 2010/0312145 A1
(43) Pub. Date: Dec. 9, 2010

(54) DEVICE FOR THE FUNCTIONAL DIAGNOSIS OF VESTIBULAR REFLEX ARCS USING MYOGENIC POTENTIALS

(76) Inventors: Arneborg Ernst, Berlin (DE); Dietmar Basta, Brieselang (DE)

Correspondence Address: JOYCE VON NATZMER;PEQUIGNOT + MYERS LLC, 200 Madison Avenue, Suite 1901, New York, NY, 10016, US

(21) Appl. No.: 12/812,356

(22) PCT Filed: Feb. 23, 2009
(86) PCT No.: PCT/DE09/00264

§ 371 Date: Aug. 23, 2010

(30) Foreign Application Priority Data
Feb. 22, 2008 (DE) 10 2008 011046.9
Apr. 7, 2008 (DE) 10 2008 017 994.9
May 5, 2008 (DE) 10 2008 022 148.1
Jul. 15, 2008 (EP) 08075639.8

(51) Int. Cl. A61B 5/103

(52) U.S. Cl. 600/587

(57) ABSTRACT

The invention relates to a device for the detection of at least one vestibular evoked myogenic potential and the use of this device for the diagnosis of the otolithic organs, particularly for the measurement and/or evaluation of vertigo phenomena in a patient. The use particularly relates to a device for the functional diagnosis of acoustically, mechanically, or electrically evoked vestibular reflexes.

The invention relates to a device for the detection of at least one vestibular evoked myogenic potential and the use of said device for the diagnosis of the otolith organs, particularly for the measurement and/or evaluation of vertigo phenomena in a patient, and more specifically, the invention relates to the use of a device for the functional diagnosis of acoustically, mechanically or electrically evoked vestibular reflexes.

Vertigo is one of the most common complaints in medical practice. Patients are using the term “vertigo” for a number of sensory perceptions, such as rotary or postural vertigo, wobbly gait and/or postural instability, weakness and oscillopsia. The prevalence increases with age, so that vertigo is the most commonly complained symptom of disease in those more than 80 years of age. Vertigo in medical terms refers to a subjective sensation of spinning or unsteadiness or a feeling of impending unconsciousness. Vertigo in medical terms is defined as a perceived apparent motion between self and the environment. Vertigo frequently arises from contradictory information from the sensory organs involved in the sensation of equilibrium, such as eyes, organs of equilibrium of the inner ears as well as muscle and joint receptors. The organ of equilibrium in the inner ear is a sensory apparatus for rotary and linear acceleration and closely related to reflexes.

Linear acceleration is detected in the macula sacculi and macula utriculi arrayed in horizontal and vertical planes. The sensory hairs of these receptors are embedded in a matrix weighted by crystal grains, so-called otoliths. Upon acceleration in the plane of the macula, the matrix lags behind as a result of its inertia, giving rise to a deflection of the sensory hairs. Also, by virtue of gravity, the position of the head in space can be determined by these receptors.

Rotary acceleration is detected by the semicircular canals—three interconnected, mutually perpendicular, annular vessels including lymphatic fluid and sensory hairs. Due to the rotary acceleration in the plane of the respective semicircular canal, the endolymphatic fluid lags behind the moving skull bone as a result of mass inertia forces. A gelatinous membrane stretching across the fluid aids in transferring the fluid movement to sensory hairs to deflect the latter. Persisting rotary motion leads to a concomitant movement of the endolymph, the sensory stimulus is reduced and eventually drops towards zero as semicircular canal and endolymph move at the same velocity, thereby resulting in habituation. When the rotary motion ceases the fluid will continue to rotate and evokes the impression of an opposite rotation. The reflex response cannot be suppressed even if the eye can see the actual movement. The discrepancy produced by the sensory organs creates confusion or disorientation.

Vertigo can have many causes. In contrast to functional disorders of the semicircular canals in the organ of equilibrium, which can be determined with great certainty merely by assessing nystagmi under thermal stimulation, little is known as yet about diagnostics of the otolith organs. Presumably, a large number of unclear, persisting vertigo symptoms can be attributed to a non-diagnosed lesion of the otolith organs (utricle and saccule).

As for saccular function diagnostics, attempts are currently being made to establish a method that utilizes stimulation in the form of low-frequency acoustic stimulation, wherein the vestibulocollic reflex is triggered, the sensory component of which represents the saccule. The motor component can be detected by measuring a myopotential on, for example, the head/neck musculature. This response to acoustic signals is independent of the human hearing ability. The test offers the option of separately examining the saccular function on each side of the body. For stimulation, acoustic stimuli (clicks or tones) of high intensity (usually >95 dB) are presented via air or bone conduction. The myopotential is specifically referred to as “vestibular evoked myogenic potential” (VEMP). The latency and amplitude of the response are used for evaluation. A sufficient muscle tone is an essential precondition for measurement because the activity of the saccular receptor epithelial cells inhibits the ipsilateral muscles. That is, no VEMP will be generated in the absence of sufficient muscle activity. Muscle tone is understood to be a tension condition of the muscles and is caused by permanent contraction. Its intensity is proportional to the contraction intensity. The amplitude of the VEMPs is highly dependent on the intensity of the muscle tone during measurement and on the patient's age and sex. Disadvantageously, the muscle tone is never close to constancy during measurement so that the results of potential averaging are always somewhat distorted. The known devices, which present a click or tone stimulus and record the muscular activity, are not suitable for performing standardized VEMP measurements as a function of a patient's age and sex. In known devices the muscle tone required for measurement is generated in an arbitrary and usually non-uniform fashion by contracting the muscle. As a result, objective, qualitative and quantitative assessment of the saccular function is not possible in clinical practice.

The object of the invention was therefore to provide means and uses that do not have the disadvantages of the prior art and allow qualitative and quantitative assessment of in particular the saccular functions and preferably integrated evaluation of the VEMP signals with reference to standard values taking into account the patient's age, sex and muscle tone during measurement.

Surprisingly, the object of the invention can be accomplished by means of a device for detecting at least one vestibular evoked myogenic potential (VEMP) in a patient, said device comprising at least one active electrode, a reference electrode, a grounding electrode, an acoustic, mechanical and/or electrical signal generator and a feedback system, in particular a galvanometer indicating the muscle tone or a pressure sensor indicating the pressure. It was quite surprising to find that a device constituted of the above-mentioned three electrodes, an acoustic, mechanical and/or electrical signal generator and a feedback system, in particular a galvanometer indicating the muscle tone or a pressure sensor indicating the pressure, is capable of solving the problem according to the invention.

A feedback system in the meaning of the invention is any means that transmits information on the muscle tone to the persons involved in the measurements. The system can be selected in such a way that either the patient receives a direct feedback regarding the muscle tension or the medical staff gathers this information and thus can ask the patient e.g. to modify, i.e. in particular increase or reduce, the muscle tension. A particularly effective feedback system is designed in such a way that the relevant data are displayed in the patient's field of vision during measurement. Such data can be, for example, a displayed voltage or pressure generated by activation of a muscle. Of course, it is also possible that a sensor receives information on the muscle tone intensity in the form of a pressure. This may include all information that can be reflected in a pressure sensor as a result of muscular activity.

Some of the terms used in the context of the invention will be defined below:

The acoustic signal generator is in particular characterized in that it causes vibrations in parts of the body, especially in the region of the head.

The mechanical signal generator can also be characterized in that movable parts are used to transfer mechanical energy to parts of the body, especially in the region of the head.

The electrical signal generator is characterized in that it transfers electricity to parts of the human body.

The galvanometer indicates the change in the amount of existing electricity without calibration. The initial value (zero) can therefore be adjusted individually in each measurement.

For measurement, the active electrode is applied to a muscle of the patient. In a preferred embodiment of the invention the reference and grounding electrodes are attached to a non-muscular part of the body, such as sternum or forehead. In a particularly preferred embodiment of the invention the muscle is a muscle of the neck, extremities or eyes.

In another preferred embodiment of the invention the required muscle tone of at least one muscle on the side of the body to be examined is generated by contraction or movement. The resulting neck muscle potential is conducted via the electrodes and supplied to a processing unit. In another preferred embodiment of the invention the processing unit may include a measurement amplifier with integrated filter function.

The feedback system allows a patient to control the intensity and uniformity of muscle tension either independently or by following instructions by the personnel present during measurement.

In a preferred embodiment of the invention the muscle tone is displayed in the form of a perceptible, preferably optical, acoustic or vibrotactile, signal on a patient display. In this way, the muscle activity or muscle tension can either be held constant or modified to a desired extent by the patient in a direct and immediate manner.

In another preferred embodiment of the invention the feedback system is a system that visualizes the pressure generated when rotating or tilting the head, the pressure sensor being an essentially U-shaped, tubular element that can be filled with gas and/or liquid and can be placed e.g. around the neck of a patient during measurement. In a preferred fashion the tubular element worn around the neck is configured such that it comprises a pressure sensor/pressure transducer that measures the arising pressure and simultaneously displays it on a display—in particular a display visible to the patient—during measurement. In a preferred embodiment of the invention the patient, using e.g. his or her chin, presses the U-shaped gas- or liquid-filled element during measurement, preferably the tube placed around his or her neck. The pressure arising in the tube is measured by a pressure transducer or pressure sensor situated therein and displayed on a display, preferably situated in the patient's field of vision, so as to allow e.g. control of a sufficient muscle tone. In another preferred embodiment of the invention, a potential is detected and conducted via the electrode, preferably in a processing unit. It is of course also possible to attach the tubular element to at least one side of the patient's head in order to regulate the muscle tone by pressing against an immobile object. When performing the measurement in a horizontal position, muscle tension can be achieved e.g. by raising the head. In this case, the tubular element is placed beneath the head in horizontal position, and the pressure decrease during raising the head is used as quantity to be regulated.

The invention also relates to the use of said device for generating, measuring and/or assessing at least one vestibular evoked myogenic potential or in otolith organ diagnostics to detect e.g. various forms of vertigo.

Diagnosis or finding a diagnosis in the meaning of the invention relates to generating intermediate results required for establishing a diagnosis, so that a final diagnosis is possible through sophisticated intellectual activity and comparison with additional data. Thus, by using the device of the invention, a technician can create a working basis for subsequent diagnostic work of a physician.

In another preferred embodiment, diagnosis or finding a diagnosis relates to all medical methods of examination.

Furthermore, the device according to the invention is preferably used together with a processing unit wherein preferred essential diagnostic steps can be carried out. For example, this applies in those cases where the evaluation of measured data in preferred embodiments of the invention is performed using a microcontroller which determines the smallest and the largest voltage value of the VEMP within a time window following the stimulus. In a preferred variant of the invention the points in time of the above two data values (latencies) and their difference in magnitude (amplitude) are compared with age- and sex-related standard values by a microprocessor in the processing unit, taking into account the intensity of the existing muscle tone. To assess the value of the measured amplitude, the value of the muscle tone is divided by the amplitude value in order to determine in a particularly preferred embodiment of the invention a diagnostic intermediate result which in turn is used in establishing a diagnosis. In another preferred embodiment the amplitude value is divided by the muscle tone value to determine the diagnostic intermediate result. In the former case, when measuring the neck muscles and using acoustic stimulation, the influence of age on the calculated quotient can be represented by the function y=0.0548x+2.6887, where y is the quotient of muscle tone/amplitude (each in μV) and x represents the age in years. The intermediate result is to be considered as pathological when the quotient determined in the patient under examination is higher than that calculated using the function described above. In contrast, when exceeding 18 ms for the first peak of the VEMP or 27.6 ms (males) and 25.6 ms (females), respectively, for the second peak of the VEMP, the latencies are excessively long. The above data are based on a linear regression of the upper 90% confidence interval of the amplitudes or on the mean value added with the double standard deviation of the latencies in a healthy normal population.

The level of exceedance of the normal values for amplitude and latencies represents a measure of the degree of functional impairment of the vestibulocollic reflex. In this way, diagnostic quantitative statements as to the function of the saccule are possible for the first time. If, despite an existing age-dependent minimum muscle tone, a VEMP cannot be detected, a failure of the saccular function or other component of the vestibulocollic reflex is present (qualitative statement). During measurement the device compares the present muscle tone with the internally stored age-dependent minimum muscle tone (93.5 μV (20-40 years), 104.8 μV (41-60 years), 110.8 μV (60-76 years)) and, if the muscle tone falls below this value, gives out a warning that measurement is not possible. The validity of the qualitative statement is significantly increased in this way.

In a preferred embodiment the neck muscle where the resulting potential is detected and conducted via the electrode is the sternocleidomastoid muscle. Measurement in this muscle can proceed in a particularly reliable and effective manner.

In another likewise preferred embodiment of the invention the muscle tone can be recorded continuously between the VEMP measurements by measuring the potential and presented to the patient in the form of a signal that can be perceived by the patient. The potential arising in the muscle is proportional to the intensity of the muscle tone. To measure the VEMPs, the patient is subjected to short acoustic stimuli using an air or bone conduction sound generator, or the saccule or balance nerve is stimulated electrically. The VEMPs are preferably conducted via the electrodes and supplied to the processing unit.

In another preferred variant of the invention a plurality of VEMPs can be averaged in the processing unit so as to increase the signal quality (signal-to-noise ratio). Also, in another preferred embodiment of the invention, assessment of the measured data can proceed automatically. In another preferred embodiment of the invention, at least one acoustically evoked brain potential can be measured particularly after rearranging the active electrode and preferably the reference electrode.

Quite surprisingly, the teaching according to the invention can be used to overcome the drawbacks of the prior art. The teaching according to the invention combines different elements to achieve the overall technical success. The invention fills the long-standing need for standardized VEMP measurements. Despite many efforts, a solution to this problem has not been achieved as yet. Also, the simplicity of the inventive solution of the problem is indicative of inventive activity because it is precisely simple solutions hitherto unknown in the art that are more difficult to implement than complex solutions. The teaching according to the invention represents an achievement that rationalizes development, wherein simplification results in savings of time, materials, work steps and cost, and enhanced reliability is possible by elimination of flaws.

The teaching according to the present application is remarkable for the following features:
Departure from conventional technologies
New field of problems
Existence of a long-unsatisfied, urgent need for the solution of the problem solved by the invention
Hitherto vain efforts in the art
Simplicity of a particular solution indicates inventive activity, especially as it replaces more complicated teachings
Development in scientific technology has proceeded in a different direction
Achievement that rationalizes development
Erroneous ideas in the art on the solution of the problem at issue (prejudice)
Technical progress, e.g. improvement, performance enhancement, lower expense, savings of time, materials, work steps, cost or raw materials difficult to obtain, enhanced reliability, elimination of flaws, superior quality, maintenance-freedom, greater efficiency, higher yield, expansion of the technical scope, provision of a further means, creation of a second approach, creation of a new field, first-time solution of a problem, reserve means, alternatives, scope for rationalization, automation or miniaturization, or enrichment of the range of available drugs
Fortunate choice because one has been selected out of a variety of possibilities, the result of which has not been predictable, this therefore being a patentable fortunate choice
Errors in the technical literature or highly contradictory representation of the subject matter of the invention
Young field of technology
Combination invention, i.e. several known elements have been combined to achieve a surprising effect
Issue of licenses
Praise in the art
Economic success.

In particular, the advantageous embodiments of the invention have at least one or more of the advantages mentioned above.

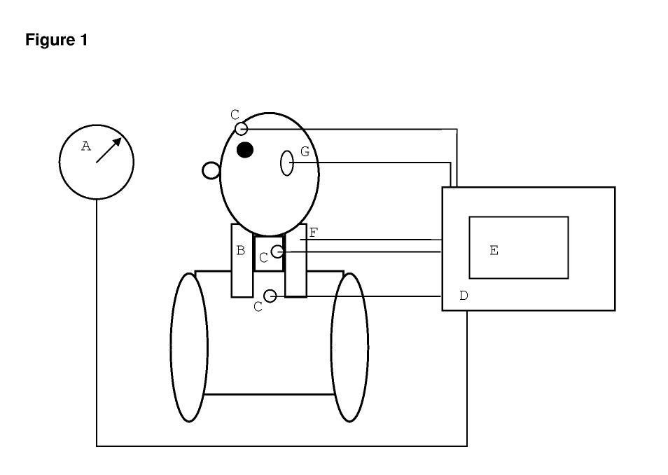

Without intending to be limiting, the invention will be explained in more detail with reference to FIG. 1 and the examples. FIG. 1 shows a schematic representation of a preferred embodiment of the invention during measurement:

FIG. 1: Schematic representation of the use of the invention during a measurement.

A—Patient display

B—Pressurized tube

C—Electrodes

D—Processing unit

E—Display

F—Output of pressure sensor in pressurized tube

G—Connection of an acoustic signal transducer

Example of a VEMP Measurement:

Single electrodes are attached to the sternocleidomastoid muscle, vertex and sternum of a patient having vertigo symptoms. An acoustic signal transducer is inserted in the auditory canal on the side to be examined and where the electrode is fixed on the sternocleidomastoid muscle. The patient now turns the head towards the contralateral shoulder, thereby increasing the muscle tone of the sternocleidomastoid muscle on the side to be examined. In addition, the display of the galvanometer now comes into the patient's field of vision, the pointer deflection of the galvanometer informing the patient whether the muscle tone in relation to the age of the patient is sufficient for a functional test of the saccule. If the galvanometer signals sufficient muscle tone, the measurement is started in such a way that the acoustic signal transducer emits 5 tones per second at a frequency of 500 Hz and a loudness level of 95 dB. Stimulation is interrupted as soon as the galvanometer reading is no longer in the demanded range. The potentials arising in the muscle are recorded within a time window of 100 ms after the stimulus and averaged over 130 repetitions. The averaged potential (VEMP) in the male patient under examination (36 years of age) has first and second peak latencies of 14 ms and 24 ms, respectively. These values are in the normal range. With a value of 180.1 μV, the magnitude of the potential (amplitude) in relation to the muscle tone intensity during measurement and age of the patient is regarded as normal. Using these intermediate results, a physician now would place the focus of differential diagnostics on central nervous aspects or functional diagnostics of other peripheral equilibrium receptors.

Example of Measurement of Acoustically Evoked Brain Potentials:

Single electrodes are attached to the vertex, mastoid and sternum of a patient with impaired hearing. An acoustic signal transducer is inserted in the auditory canal on the side to be examined and where the electrode is fixed on the mastoid. The patient is asked to sit quietly or remain in horizontal position. The acoustic signal transducer now emits 20 acoustic stimuli per second with a defined frequency spectrum and a loudness level of 70 dB.

The electrodes conduct the brain potentials resulting from the acoustic stimulation. The potentials are recorded within a time window of 15 ms after the stimulus and averaged over 2000 repetitions. With 1.8 ms for wave I, 2.9 ms for wave II, 3.8 ms for wave III, 5.0 ms for wave IV, and 5.8 for wave V, the latencies of the averaged brain potentials of the patient under examination are in the normal range. Using these intermediate results, a physician now would place the focus of differential diagnostics on cochlear functional disorders.

1. A device for detecting at least one vestibular evoked myogenic potential (VEMP) in a patient,
wherein
the device comprises at least one active electrode, a reference electrode, a grounding electrode, an acoustic, mechanical and/or electrical signal generator and a feedback system.

2. The device according to claim 1,
wherein
a muscle tone and/or a visualized pressure is displayed in form of a perceptible, signal on a patient display.

3. The device according to claim 16,
wherein
the muscle tone is measured using a pressure sensor, said pressure sensor being an essentially U-shaped, tubular element that can be filled with gas and/or liquid.

4. The device according to claim 3,
wherein
the pressure arising in the tubular element is measured with the pressure sensor and can be displayed in a patient display.

5. A method for generating, measuring and/or evaluating at least one vestibular evoked myogenic potential comprising providing the device of claim 1 and generating, measuring and/or evaluating said at least one vestibular evoked myogenic potential via said device.

6. A method for obtaining intermediate results as a basis of otolith organ diagnostics comprising providing the device of claim 1 and obtaining said intermediate results as a basis of otolith organ diagnostics via said device.

7. A method for obtaining intermediate results for establishing a diagnosis of vertigo, especially rotary, postural, lift, movement and/or non-systematic vertigo comprising providing the device of claim 1 and obtaining said intermediate results for establishing said diagnosis of vertigo via said device.

8. A method for detecting intermediate results in finding a diagnosis regarding a function of vestibular reflexes comprising providing the device of claim 1 and detecting said intermediate results for finding a diagnosis regarding the function of vestibular reflexes via said device.

9. The method of claim 7, wherein
the muscle tone is measured using a pressure sensor, said pressure sensor being an essentially U-shaped, tubular element that can be filled with gas and/or liquid and the muscle tone is recorded continuously between the VEMP measurements by measuring the potential and presented to the patient instead of and/or in addition to the pressure measurement in the tube in the form of a signal that can be perceived by the patient.

10. The method of claim 10, wherein acoustic, mechanical or electrical stimuli are presented to the patient for the measurement of VEMPs.

11. The method of claim 10, wherein the smallest and largest voltage values of the VEMP are determined within a time window following the signal.

12. The method of claim 10, wherein the points in time of the smallest and largest voltage values of the VEMP and their difference in magnitude are compared and matched with age- and/or sex-related standard values, taking into account the intensity of the muscle potential.

13. The method of claim 7, wherein
the influence of age on the calculated quotient is represented by the function y=0.0548x+2.6887, where y is the quotient of muscle tone/amplitude in μV and x represents the age in years.

14. The method of claim 13, wherein an intermediate result is considered pathological when the quotient determined in the patient is higher than that calculated using the function y=0.0548x+2.6887.

15. A method for measuring acoustically evoked brain potentials comprising providing the device of claim 1 and measuring acoustically evoked brain potentials via said device.

16. A device according to claim 1,
wherein said feedback system is a galvanometer indicating muscle tone and/or a pressure sensor indicating pressure.

17. The device according to claim 3,
wherein the perceptible signal is an optical, acoustic and/or vibrotactile signal.

18. The method of claim 7 wherein the vertigo is rotary, postural, lift, movement and/or non-systematic vertigo.
